(19) United States
(12) Patent Application Publication
(10) Pub. No.: US 2023/0264187 A1
(43) Pub. Date: Aug. 24, 2023

(54) Apparatus, Methods, and Systems for Fluid Separation

(71) Applicant: Laboratory Corporation of America Holdings, Burlington, NC (US)

(72) Inventor: Pratistha Ranjitkar, Seattle, WA (US)

(21) Appl. No.: 18/174,334

(22) Filed: Feb. 24, 2023

Related U.S. Application Data
(60) Provisional application No. 63/313,476, filed on Feb. 24, 2022.

(51) Int. Cl. B01L 3/00

(52) U.S. Cl. CPC B01L 3/502; B01L 3/5635; B01L 2200/026; B01L 2200/04; B01L 2300/0864

(57) ABSTRACT

The present disclosure relates to an apparatus, method, and system for separating a fluid sample. The apparatus for separating a fluid sample may include a receiving chamber comprising a first opening and a second opening. The first opening may have a larger diameter than the second opening. The apparatus may include a channel extending from the second opening of the receiving chamber. The channel may include a proximal end adjacent the receiving chamber and a distal end. The distal end of the channel may bifurcate into a first branch and a second branch. The first branch and the second branch can be inserted into a first container and a second container through a cap having an orifice to separate a fluid sample into the first container and the second container.

### CROSS-REFERENCE TO RELATED APPLICATION

This application claims the benefit of and priority to U.S. Provisional Application No. 63/313,476, filed Feb. 24, 2022, which is incorporated herein by reference in its entirety for all intents and purposes.

### FIELD

This disclosure relates to separating a fluid sample for analysis. More specifically, this disclosure provides an apparatus, system, and method for separating a fluid sample into portions (e.g., equal amounts) in a sanitary and efficient process for urine analysis.

### BACKGROUND

Urine analysis is a common medical test that can provide health-related information about the person or animal that provided the urine sample. A urine sample can be analyzed to diagnose and/or monitor disease conditions. A urine sample can also be analyzed to determine whether a person has consumed illegal or banned substances or to detect the presence of biomolecules that indicate whether a person has developed cancer, or has any of a number of infectious diseases.

A patient typically provides one or more urine samples to a physician or other medical professional. For example, a urine sample can be collected in a container over a 24-hour period. The urine samples are sent (e.g., through a courier service) to a medical laboratory for analysis. Trained medical laboratory technologists or physicians at the medical laboratory then conduct the necessary tests on the urine samples, and subsequently send the results back to the physician. Tests in a medical laboratory are often conducted in batches, with several urine samples from different people being analyzed together.

A number of problems arise with this process when multiple urine samples are needed. A patient may be required to collect urine samples over multiple 24-hour periods due to different preservative requirements for a panel to test, resulting in delayed testing and diagnosis. Additionally, there is potential for urine samples to be mixed up, misplaced, or tampered with, either at the laboratory or during delivery, thereby reducing the accuracy and reliability of the analysis.

Therefore, methods and systems are needed for collecting urine samples with less delays for urine sample collection and reduced risks of cross-contamination.

### SUMMARY

Covered embodiments of the present disclosure are defined by the claims, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification, any or all drawings and each claim.

In some embodiments, the present disclosure provides an apparatus for separating a fluid sample. The apparatus includes a receiving chamber comprising a first opening and a second opening. The first opening may have a larger diameter than the second opening. The apparatus includes a channel extending from the second opening of the receiving chamber. The channel may include a proximal end adjacent the receiving chamber and a distal end. The distal end of the channel bifurcates into a first branch and a second branch. In some embodiments, the receiving chamber comprises a truncated cone, wherein the truncated cone has an interior volume configured to receive the fluid sample. In some embodiments, the diameter of the receiving chamber continually decreases from the first opening to the second opening. In some embodiments, the first branch and the second branch extend in opposing directions from the channel. In some embodiments, the first branch and the second branch are disposed at an acute angle from a long axis of the channel. In some embodiments, a diameter of the channel from the proximal end to the distal end is constant. In some embodiments, the distal end of the channel, the first branch, and the second branch form a junction, wherein the junction is configured to separate the fluid sample into the first branch and the second branch. In some embodiments, a diameter of the first branch and the second branch is less than the diameter of the channel. In some embodiments, the fluid sample comprises urine. In some embodiments, the apparatus includes a first leg extending from the first branch and a second leg extending from the second branch. In some embodiments, the first leg and the second leg are parallel to the channel. In some embodiments, the apparatus includes a first cap for receiving the first branch and a second cap for receiving the second branch. In some embodiments, the first cap and the second cap each comprise an orifice configured to receive the first branch and the second branch. In some embodiments, the first cap and the second cap include a plastic disposed over the orifice, wherein the plastic provides a seal when the first branch and the second branch are each inserted into the first cap and the second cap through the orifice. In some embodiments, the first cap and the second cap are self-sealing caps optionally fitted to attach to and secure the apparatus to a first orifice on a first container and a second orifice on a second container, respectively.

In some embodiments, the present disclosure provides a system for separating a fluid sample. The system includes an apparatus. The apparatus includes a receiving chamber comprising a first opening and a second opening. The first opening may have a larger diameter than the second opening. The apparatus includes a channel extending from the second opening of the receiving chamber. The channel may include a proximal end adjacent the receiving chamber and a distal end. The distal end of the channel bifurcates into a first branch and a second branch. The system includes a first container comprising a first cap and a second container comprising a second cap. In some embodiments, the diameter of the receiving chamber continually decreases from the first opening to the second opening. In some embodiments, the first branch and the second branch are disposed at an acute angle from a long axis of the channel. In some embodiments, a diameter of the channel from the proximal end to the distal end is constant. In some embodiments, the distal end of the channel, the first branch, and the second branch form a junction, wherein the junction is configured to separate the fluid sample into the first branch and the second branch. In some embodiments, the apparatus further comprises a first leg extending from the first branch and a second leg extending from the second branch. In some embodiments, the first leg and the second leg are parallel to the channel. In some embodiments, the first cap and the second cap each comprise an orifice configured to receive the first leg and the second leg. In some embodiments, the first cap and the second cap include a plastic disposed over the orifice, wherein the plastic provides a seal when the first leg and the second leg are each inserted into the first cap and the second cap through the orifice. In some embodiments, the first cap and the second cap are self-sealing caps optionally fitted to attach to and secure the apparatus to a first orifice on the first container and a second orifice on the second container, respectively.

In some embodiments, the present disclosure provides a method for separating a fluid sample. The method includes providing an apparatus. The apparatus includes a receiving chamber comprising a first opening and a second opening. The first opening may have a larger diameter than the second opening. The apparatus includes a channel extending from the second opening of the receiving chamber. The channel may include a proximal end adjacent the receiving chamber and a distal end. The distal end of the channel bifurcates into a first branch and a second branch. The method includes providing a first container and a second container.

The first container includes a first cap and the second container includes a second cap. The method includes inserting the first branch through the first cap into the first container and the second branch through the second cap into the second container. The method includes providing a fluid sample to the receiving chamber of the apparatus and separating the fluid sample into the first container and the second container. In some embodiments, inserting the first branch into the first cap and inserting the second branch into the second cap comprises puncturing a sealing film disposed over an orifice of the first cap and the second cap. In some embodiments, the method includes collecting a fluid sample. In some embodiments, the fluid sample is separated into the first container via the first branch and the second container via the second branch. In some embodiments, the method includes removing the first branch and the second branch from the first cap and the second cap after the fluid sample is collected. In some embodiments, the method includes replacing each of the first cap and the second cap with a tamper-resistant seal cap.

In some embodiments, the present disclosure provides an apparatus for separating a fluid sample. The apparatus includes: a first-fold section comprising: a portion of a receiving chamber; a portion of a channel extending from a distal end of the receiving chamber, wherein the channel bifurcates into a portion of a first branch and a second branch; a second-fold section comprising: a second portion of the receiving chamber; a second portion of the channel extending from a distal end of the receiving chamber, wherein the channel bifurcates into a second portion of the first branch and the second branch; and a middle-fold section disposed between the first-fold section and the second-fold section, wherein the middle-fold section comprises a first end cap and a second end cap; wherein the first-fold section and the second-fold section are configured to fold inward toward the middle-fold section. In some embodiments, the apparatus includes a first hinge between the first-fold section and the middle-fold section, and a second hinge between the second-fold section and the middle-fold section. In some embodiments, the first-fold section is configured to be removably attached to the second-fold section. In some embodiments, the portion of the receiving chamber of the first-fold section corresponds to the second portion of the receiving chamber of the second-fold section to form the receiving chamber, the portion of the channel of the first-fold section corresponds to the second portion of the channel of the second-fold section to form the channel, and the portion of the first branch and second branch of the first-fold section corresponds to the second portion of the first branch and second branch of the second-fold section to form the first branch and second branch. In some embodiments, the portion of the receiving chamber of the first-fold section and the second portion of the receiving chamber of the second-fold section increase in depth and width from a first end to a second end. In some embodiments, the first-fold section and the second-fold section comprise an interior-facing sidewall extending from a top face to the middle-fold section. In some embodiments, the interior-facing sidewall is curved. In some embodiments, wherein the interior-facing sidewall of the first-fold section includes a portion of a first through hole and a second through hole, wherein the interior-facing sidewall of the second-fold section includes a second portion of the first through hole and the second through hole. In some embodiments, each of the first end cap and the second end cap comprise: a cylindrical insert member comprising an orifice; an annular groove surrounding the cylindrical insert member; and an outer wall. In some embodiments, the cylindrical insert member includes bottom region that tapers to the orifice. In some embodiments, the middle-fold section comprises a hemispherical body. In some embodiments, a top face of the first-fold section includes a plurality of grooves and a top face of the second-fold section includes a plurality of protrusions.

In some embodiments, the present disclosure provides a system for separating a fluid sample. The system includes an apparatus comprising: a first-fold section comprising: a portion of a receiving chamber; a portion of a channel extending from a distal end of the receiving chamber, wherein the channel bifurcates into a portion of a first branch and a second branch; a second-fold section comprising: a second portion of the receiving chamber; a second portion of the channel extending from a distal end of the receiving chamber, wherein the channel bifurcates into a second portion of the first branch and the second branch; and a middle-fold section disposed between the first-fold section and the second-fold section, wherein the middle-fold section comprises a first end cap and a second end cap; wherein the first-fold section and the second-fold section are configured to fold inward toward the middle-fold section to enclose the middle-fold section. The system further includes a first container and a second container. In some embodiments, each of the first end cap and the second end cap comprise: a cylindrical insert member comprising an orifice; an annular groove surrounding the cylindrical insert member; and an outer wall. In some embodiments, the cylindrical insert member includes a bottom wall that tapers to the orifice. In some embodiments, the cylindrical insert member is configured to be inserted within the first container and the second container. In some embodiments, the apparatus further comprises: a first hinge between the first-fold section and the middle-fold section; and a second hinge between the second-fold section and the middle-fold section. In some embodiments, a top face of the first-fold section includes a plurality of grooves and a top face of the second-fold section includes a plurality of protrusions. In some embodiments, the first-fold section and the second-fold section comprise an interior-facing sidewall extending from a top face to the middle-fold section, wherein the interior-facing sidewall is curved.

In some embodiments, the present disclosure provides a method for separating a fluid sample. The method includes providing an apparatus. The apparatus comprises: a first-fold section comprising: a portion of a receiving chamber; a portion of a channel extending from a distal end of the receiving chamber, wherein the channel bifurcates into a portion of a first branch and a second branch; a second-fold section comprising: a second portion of the receiving chamber; a second portion of the channel extending from a distal end of the receiving chamber, wherein the channel bifurcates into a second portion of the first branch and the second branch; and a middle-fold section disposed between the first-fold section and the second-fold section, wherein the middle-fold section comprises a first end cap and a second end cap; wherein the first-fold section and the second-fold section are configured to fold inward toward the middle-fold section to enclose the middle-fold section. The method includes providing a first container and a second container. The method includes attaching the first end cap to the first container. The method includes attaching the second end cap to the second container. The method includes attaching the first-fold section to the second-fold section. The method includes providing a fluid sample to the receiving chamber of the apparatus. The method includes separating the fluid into the first container and the second container. In some embodiments, attaching the first end cap to the first container comprises inserting a cylindrical insert member of the first end cap into a mouth of the first container. In some embodiments, the method further comprises collecting a fluid sample. In some embodiments, the sample is separated into the first container via the first branch and the second container via the second branch. In some embodiments, the method further comprises removing the apparatus after the fluid sample is collected. In some embodiments, the method further comprises sealing the first container and the second container.

Further aspects, objects, and advantages will become apparent upon consideration of the detailed description.

### BRIEF DESCRIPTION OF THE DRAWINGS

FIG. 1 illustrates a perspective view of an apparatus for separating a fluid sample according to some embodiments.

FIG. 2 illustrates another perspective view of an apparatus for separating a fluid sample according to some embodiments.

FIG. 3 illustrates a system for collecting and separating a fluid sample according to some embodiments.

FIG. 4 provides a flow diagram of a method of collecting a fluid sample according to some embodiments.

FIGS. 5A-5C illustrate perspectives view of an apparatus including a trifold design for separating a fluid sample according to some embodiments.

FIGS. 6A-6I illustrate multiple views of a system for separating a fluid into containers using an apparatus including a trifold design according to some embodiments.

FIG. 7 provides a flow diagram of a method of separating a fluid sample according to some embodiments.

### DETAILED DESCRIPTION OF SPECIFIC EMBODIMENTS

The present disclosure describes a number of embodiments related to an apparatus, system, and method for collecting a fluid sample for analysis. In some instances, the apparatus, system, and method described herein separate a fluid sample into two portions (e.g., equally) to provide two samples for analysis. The apparatus for separating a fluid sample may include a receiving chamber to receive a sample (e.g., urine). The apparatus may include a channel extending from an opening of the receiving chamber. The channel is configured to receive the fluid sample from the receiving chamber. The distal end of the channel may bifurcate into a first branch and a second branch. The first branch and second branch can separate the sample into equal portions. For example, the first branch and the second branch can separate a urine sample into a first container and a second container via each of the branches. Additionally, the containers may include caps that are configured to receive the first branch and the second branch of the apparatus to collect a fluid sample through the cap.

In some embodiments, the apparatus for separating a fluid sample may have a trifold configuration. The apparatus may include a first-fold section, a middle-fold section, and a second-fold section. The middle-fold section may be disposed between the first-fold section and the second-fold section. In some embodiments, the first fold-section is attached to a first side of the middle-fold section and the second-fold section is attached to second side of the middle-fold section. For example, each of the first-fold section and the second-fold section may be connected to the middle fold section by a hinge. The hinge is configured to allow each of the first-fold section and the second-fold section to move inward towards the middle fold section. The first-fold section and the second-fold section can be removably attached to produce a funnel including a receiving chamber, a channel extending from the receive chamber, and a first and second branch extending from a distal end of the channel. The first-fold section and the second-fold section may each comprise a portion (e.g., corresponding half portions) of the funnel such that the first-fold section and the second-fold section form the whole parts of the funnel when attached together. The first-fold section and the second-fold section may completely enclose the middle-fold section to prevent any fluid leakage during fluid separation. The middle fold section may include a first end cap and a second end cap including an orifice to supply a fluid to a container. The first end cap and the second end cap are configured to be removably attached to a container opening. Each of the first branch and the second branch are configured to separate a fluid sample into the first end cap and the second end cap to a first container and a second container, respectively.

In some instances, patients may need to provide multiple fluid samples for analysis. For example, for 24-hour urine collection, the tests may require two urine samples collected from a patient over a 24 hour period. This typically requires two urine sample containers, one of which contains an acid preservative. Therefore, a patient may need to collect samples over two 24-hour periods to provide a urine sample for each container. Some conventional methods and systems for collecting urine includes two containers that include apertures in the body of the container for receiving a splitter device. The splitter device portions the urine sample into the two containers. However, this design of the system for collecting urine may be susceptible to leakage from the container due to the aperture in the body of the containers. In some instances, the containers may include a caustic substance (e.g., an acid preservative) which can lead to burns or injury. For example, some urine collection containers may include hydrochloric acid as a preservative for urine. Hydrochloric acid can cause severe chemical burns to the skin and mucous membranes.

The present disclosure provides an apparatus, method, and system that addresses the problems associated with conventional urine collection systems. Specifically, the present disclosure provides an apparatus, method, and system for collecting and separating a fluid sample with less delays for urine sample collection and reduced risks of cross-contamination. The system may include providing an apparatus that is configured to be inserted through the cap of a container and directly into the body of the container. The apparatus may include a first branch configured to be inserted through a first cap into a first container and a second branch configured to be inserted through a second cap into a second container for simultaneous collection of a fluid sample into two separate containers (e.g., over a single 24-hour period). This avoids including an aperture in the body of the container, which can be a cause for leakage or cross-contamination. The caps on the containers can include a seal (e.g., self-sealing or external seal film or external cap) after fluid sample collection. In some embodiments, the caps can be replaced with a tamper-resistant seal cap after fluid sample collection.

In some embodiments, an apparatus for separating a fluid sample is provided. The apparatus for separating a fluid sample may include a receiving chamber comprising a first opening and a second opening. The first opening may have a larger diameter than the second opening. The apparatus may include a channel extending from the second opening of the receiving chamber. The channel may include a proximal end adjacent the receiving chamber and a distal end. The distal end of the channel may bifurcate into a first branch and a second branch. The first branch and second branch can separate the sample into a first container and a second container.

In some embodiments, a system for collecting a fluid sample is provided. The system includes an apparatus. The apparatus for separating a fluid sample may include a receiving chamber comprising a first opening and a second opening. The first opening may have a larger diameter than the second opening. The apparatus may include a channel extending from the second opening of the receiving chamber. The channel may include a proximal end adjacent the receiving chamber and a distal end. The distal end of the channel may bifurcate into a first branch and a second branch. The first branch and second branch can separate the sample into a first container and a second container. The system includes a first container comprising a first cap and a second container comprising a second cap.

In some embodiments, a method for separating a fluid sample is provided. The method includes providing an apparatus including a receiving chamber comprising a first opening and a second opening, wherein the first opening has a larger diameter than the second opening; a channel extending from the second opening of the receiving chamber, wherein the channel comprises a proximal end adjacent the receiving chamber and a distal end; and wherein the distal end of the channel bifurcates into a first branch and a second branch. The method includes providing a first container and a second container. The method includes inserting the first branch into the first cap of the first container. The method includes inserting the second branch into the second cap of the second container. The method includes providing a fluid sample to the receiving chamber of the apparatus.

### Definitions and Descriptions

The terms “invention,” “the invention,” “this invention,” and “the present invention” used herein are intended to refer broadly to all of the subject matter of this patent application and the claims below. Statements containing these terms should be understood not to limit the subject matter described herein or to limit the meaning or scope of the patent claims below.

As used herein, the meaning of “fluid sample” includes any bodily fluid including, but not limited to, urine, blood, or saliva.

As used herein, the meaning of “a,” “an,” or “the” includes singular and plural references unless the context clearly dictates otherwise.

All ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

### EXEMPLARY EMBODIMENTS

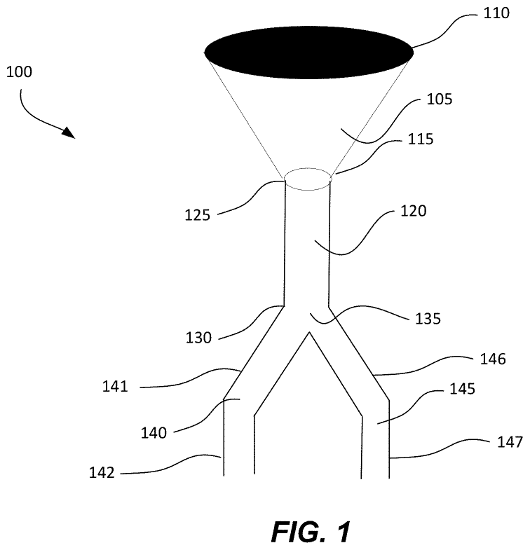

FIG. 1 illustrates an apparatus for collecting and separating a fluid sample. The apparatus 100 may include a receiving chamber 105. The receiving chamber 105 comprises an interior volume configured to receive a fluid sample. For example, a fluid sample can be poured or transferred to the receiving chamber 105 of the apparatus 100. In some embodiments, the fluid sample is urine. The receiving chamber 105 may be conical. For example, the receiving chamber 105 can have a shape of a truncated cone. In some embodiments, the receiving chamber 105 may include a first opening 110 and a second opening 115. The first opening 110 and the second opening 115 may be on opposing ends of the receiving chamber 105.

In some embodiments, the receiving chamber 105 is cone-shaped. In this embodiment, the first opening 110 may define one end of the receiving chamber 105 and the second opening 115 may define a second end of the receiving chamber 105. The diameter of the first opening 110 may be larger than the diameter of the second opening 115. In some embodiments, the ratio of the diameter of the first opening 110 to the diameter of the second opening 115 ranges from 1:1 to 20:1 (e.g., from 2:1 to 18:1, from 3:1 to 15:1, from 4:1 to 12:1, from 5:1 to 10:1, or from 2:1 to 8:1). In some embodiments, the diameter of the receiving chamber 105 decreases from the first opening 110 to the second opening 115. For example, the diameter of the receiving chamber 105 may continually decrease from the first opening 110 to the second opening 115.

The apparatus 100 may include a channel 120 extending from the second opening 115 of the receiving chamber 105. The channel 120 is configured to receive the fluid sample from the receiving chamber 105. The channel 120 includes a proximal end 125 adjacent the receiving chamber 105 and a distal end 130. In some embodiments, the diameter of the channel 120 is constant from the proximal end 125 to the distal end 130. For example, the diameter of the channel 120 may be the same at a cross section adjacent the proximal end 125 and a cross section adjacent the distal end 130. In some embodiments, the diameter of the channel 120 may vary along the entire length of the channel 120 or at discrete regions along the length of the channel 120. For example, the diameter of the channel 120 can decrease from the proximal end 125 to the distal end 130. In some embodiments, the diameter of the channel 120 may be enlarged at a central portion or at the distal end 130 of the channel 120.

The channel 120 may bifurcate into two or more branches. For example, the channel 120 can bifurcate into a first branch 140 and a second branch 145 as shown in FIG. 1. The channel 120 can bifurcate into the first branch 140 and the second branch 145 at the distal end 130 of the channel 120. The first branch 140 and the second branch 145 can separate the fluid sample from the channel 120 into two portions (e.g., two equal portions). For example, the junction 135 at which the channel 120 bifurcates distributes the fluid sample into the first branch 140 and the second branch 145. In this way, the fluid sample can be separated into each branch. The junction 135 is configured to separate the fluid sample into the first branch 140 and the second branch 145. In some embodiments, the channel 120 is a long narrow channel to facilitate splitting of the fluid sample equally into the first branch 140 and the second branch 145. For example, if a fluid sample is poured slowly into the receiving chamber and the channel has a large diameter, there may be a risk that the fluid sample is not split equally. Therefore, the channel 120 may be narrow and long such that the fluid sample is equally split.

In some embodiments, the diameter of the channel 120 is less than the diameter of the first opening 110. For example, the diameter of the channel 120 may be less than 90% of the diameter of the first opening 110 (e.g., less than 80%, less than 70%, less than 60%, less than 50%, less than 40%, less than 30%, or less than 25%). In some embodiments, the channel 120 may taper from the proximal end 125 to the distal end 130.

In some embodiments, the diameter of each of the first branch 140 and the second branch 145 is less than the diameter of the channel 120. For example, the diameter of the first branch 140 and the second branch 145 may be less than 75% of the diameter of the channel (e.g., less than 70%, less than 60%, less than 50%, less than 40%, less than 30%, less than 25%, less than 20%, less than 15%).

In some embodiments, the first branch 140 and the second branch 145 may extend in opposing directions at the junction 135. In this way, the first branch 140 and the second branch 145 can separate the fluid sample in two different directions for two separate containers. For example, the first branch 140 may be disposed at a first angle from the long axis of the channel 120 and the second branch 145 may be disposed at a second angle from the long axis of the channel 120. The first angle and the second angle may be an acute angle with respect to the long axis of the channel. The first angle and the second angle may range from 10° to 85° from the long axis of channel (e.g., from 15° to 80°, from 20° to 75°, from 25° to 70°, from 30° to 65°, or from 35° to 60°). In some embodiments, the first angle and the second angle may be the same.

In some embodiments, the first branch 140 and the second branch 145 may include a first portion 141, 146 and a second portion 142, 147. The first portion 141, 146 of the first branch 140 and the second branch 145 may extend from the junction 135. As described above, the first portion 141 of the first branch 140 may be disposed at a first angle and the first portion 146 of the second branch 145 may be disposed at a second angle. The first portion 141, 146 of each of the first branch 140 and the second branch 145 may be transverse to each other and may intersect at the junction 135. The second portion 142, 147 may extend from the distal end of the first portion 141, 146. The second portion 142, 147 of each of the first branch 140 and the second branch 145 may be parallel to each other. In some embodiments, the second portion 142, 147 may be parallel to the channel 120. The second portion 142, 147 is configured to be inserted into a cap or a container.

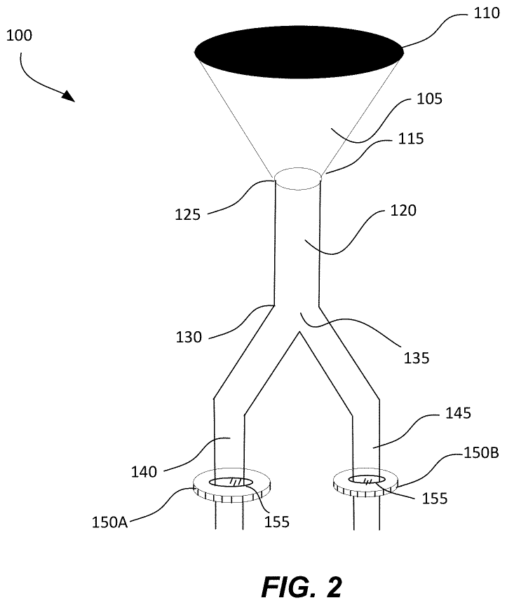

FIG. 2 shows another embodiment of an apparatus for separating a fluid sample according to some embodiments. In this embodiment, the apparatus 100 includes a first cap 150A and a second cap 150B. The first cap 150A and the second cap 150B may include an orifice 155 for receiving the first branch 140 and the second branch 145, respectively. For example, the first cap 150A may receive the first branch 140 and the second cap 150B may receive the second branch 145 through the orifice 155. In some embodiments, the first cap 150A and the second cap 150B may include a sealing film over the orifice 155. The first branch 140 and the second branch 145 may pierce the sealing film. The first branch 140 and the second branch 145 may extend through the orifice 155 to deliver the fluid sample to a container.

In some embodiments, the first cap 150A and the second cap 150B may include an aperture (not shown). The first cap 150A and the second cap 150B may include the aperture to evacuate air as the fluid sample fills the container. The aperture can facilitate the fluid sample filling the container. In some embodiments, the aperture can be located adjacent to the orifice separated by a distance. The diameter of the aperture may be smaller than the diameter of the orifice 155. For example, the diameter of the aperture may be less than 50% the diameter of the orifice (e.g., less than 60%, less than 60%, less than 70%, less than 80%, or less than 90%). In some embodiments, the first cap 150A and the second cap 150B may include a plurality of apertures.

The first cap 150A and the second cap 150B are configured to be coupled to a container. For example, the first cap 150A and the second cap 150B may include a screw thread that can mate with a screw thread on a container. The first cap 150A and the second cap 150B may include a self-sealing orifice. In some embodiments, the first branch 140 and the second branch 145 can be directly inserted into a first container and a second container via the first cap 150A and the second cap 150B. For example, the first branch 140 and the second branch 145 may be inserted into the orifice 155 in the first cap 150A and the second cap 150B such that the first branch 140 and the second branch 145 are inside a first container and a second container.

In some embodiments, the original caps of the containers can be removed from containers. The first cap 150A and the second cap 150B can be attached to the container after the original caps are removed. The first branch 140 and the second branch 145 can be directly inserted into the containers via the first cap 150A and the second cap 150B. After fluid sample collection, the first cap 150A and the second cap 150B can be removed from the containers and the original caps can be used to seal the containers.

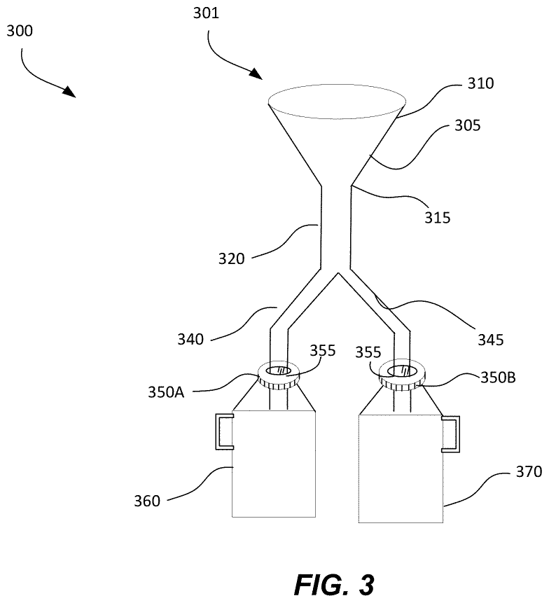

FIG. 3 shows a system for separating a fluid sample according to some embodiments. The system 300 includes an apparatus 301 for separating a fluid sample, a first container 360, and a second container 370. For example, the apparatus 301 can be any apparatus described herein. The apparatus may include a receiving chamber 305 for receiving a fluid sample. The apparatus 301 may include a channel 320 extending from the receiving chamber 305 that bifurcates into a first branch 340 and a second branch 345 for separating a fluid sample.

The first container 360 and the second container 370 may be urine specimen containers. The urine specimen containers may comprise a plastic material (e.g., polymer). For example, the urine specimen containers comprise a polymer including polypropylene or polyethylene. The urine specimen containers may have an interior volume to receive a fluid sample. For example, the container may have a volume of 3000 mL.

In some embodiments, each of the urine specimen containers may include a cap 350A, 350B. The cap can be removably attached to the first container 360 and the second container 370. In some embodiments, the caps 350A, 350B are screw caps that are configured to mate with a threaded region of the container. Each of the caps 350A, 350B may include an orifice 355. The orifice 355 may be centrally located on the cap 350A, 350B. The orifice 355 in the caps 350A, 350B is configured to receive the first branch 340 or the second branch 345 of the apparatus 301. In some embodiments, the orifice 355 in each of the caps 350A, 350B is approximately the same diameter as the diameter of the first branch 340 and the second branch 345. In embodiments where the caps 350A, 350B comprises a flexible plastic material (e.g., polyethylene), the diameter of the orifice 355 in each of the caps may be slightly smaller than the first branch 340 and the second branch 345 to provide a frictional fit.

In some embodiments, the orifice 355 of the caps 350A, 350B can be sealed after sample collection. For example, the caps 350A, 350B may include a lid that can seal the orifice 355. In some embodiments, the caps 350A, 350B may include an underlying polymer layer. As the first branch 340 and the second branch 345 are removed from the cap, the polymer layer can be pulled through the caps 350A, 350B to seal the orifice 355. In some embodiments, a sealing film can be applied over the orifice 355. The sealing film may comprise a thermoplastic polymer that seals the orifice 355. In some embodiments, the caps 350A, 350B may be self-sealing caps. Alternatively, the caps 350A, 350B can be replaced with the original caps to seal the containers.

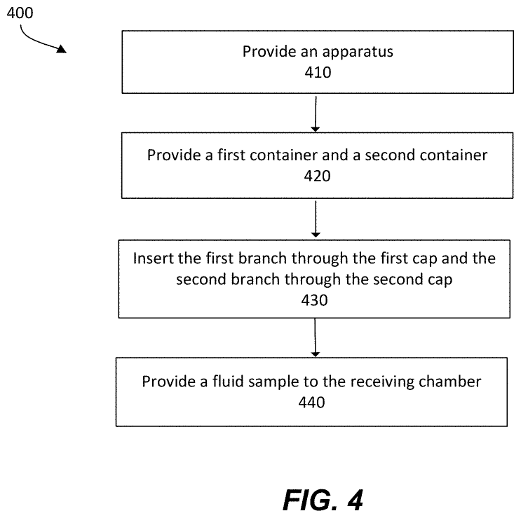

FIG. 4 provides a flow diagram of a method of separating a fluid sample according to some embodiments. The method 400 may include providing an apparatus 410. The apparatus can be any apparatus described herein. For example, the apparatus may include a receiving chamber for receiving a fluid sample. The apparatus may include a channel extending from the receiving chamber that bifurcates into a first branch and a second branch for separating a fluid sample.

The method 400 may include providing a first container and a second container 420. For example, the first container and a second container can be 24-hour urine specimen containers. The urine specimen containers may include caps. The caps on the containers may be screw caps. The screw caps can be removably attached to the containers. The screw caps may include an interior liner to prevent leakage. In some embodiments, the containers may include a handle. In some embodiments, one of the containers may include a preservative. The preservative can be an acid-based preservative. For example, the first container may include acetic acid or hydrochloric acid.

The method 400 may include inserting the first branch into the first cap and the second branch into the second cap 430. For example, the method may include inserting the first branch through the first cap into the first container and then inserting second branch through the second cap into the second container. The first branch and the second branch can be inserted into the caps simultaneously. In some embodiments, the first cap and second may include an orifice for receiving the first branch and the second branch, respectively. For example, the first cap and the second cap may include a sealed orifice for receiving the first branch and the second branch.

The distal ends of the first branch and the second branch may be inserted through the orifice of caps. In this way, the fluid sample provided to the apparatus can be separated into two portions that are provided through the first branch and the second branch to the first container and the second container. In some embodiments, the distal end of the first branch and the second branch may be angled or serrated to puncture or pierce a film disposed over the orifice of the caps.

In some embodiments, the first container and the second container may be provided with screw caps that do not include an orifice for receiving the first branch and the second branch. In these embodiments, the method may include removing the screw caps on each of the containers and attaching the caps described herein. For example, the original screw caps can be replaced with caps that include an orifice for receiving the first branch and the second branch of the apparatus.

The method includes providing a fluid sample to the receiving chamber 440. In some embodiments, the method may include collecting a fluid sample and then providing the fluid sample to the receiving chamber of the apparatus. The fluid sample may be a urine sample. The urine sample can be collected from a patient and poured into the receiving chamber of the apparatus. The urine sample travels from the receiving chamber to the channel. The urine sample is split in equal proportions into the first branch and the second branch to fill the first container and the second container via the caps.

In some embodiments, the method includes removing the first branch and the second branch from the first cap and the second cap after the fluid sample is collected. The first branch can be withdrawn from the first cap and the second branch can be withdrawn from the second cap. The orifice in the caps can be sealed. In some embodiments, the caps can be replaced with a tamper-resistant seal cap.

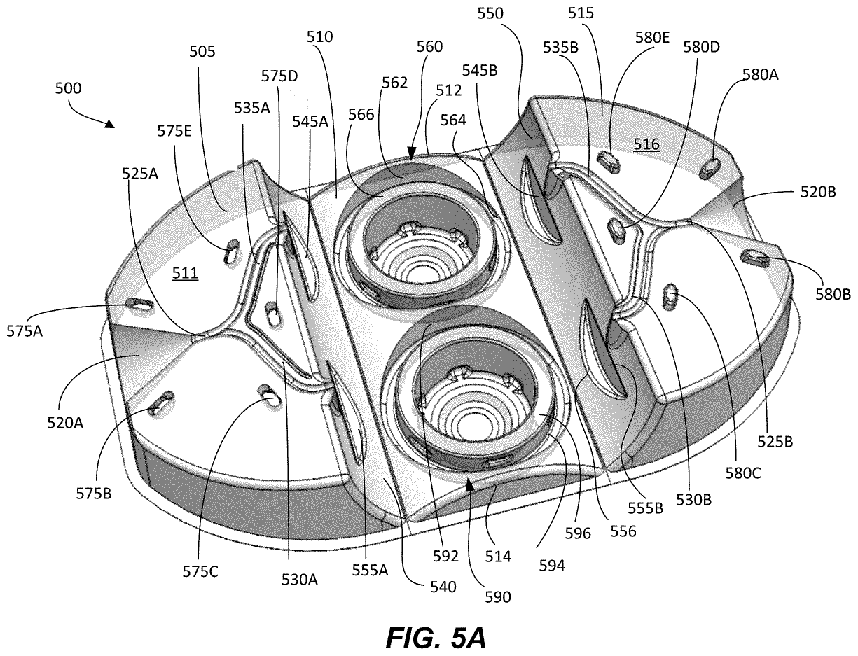

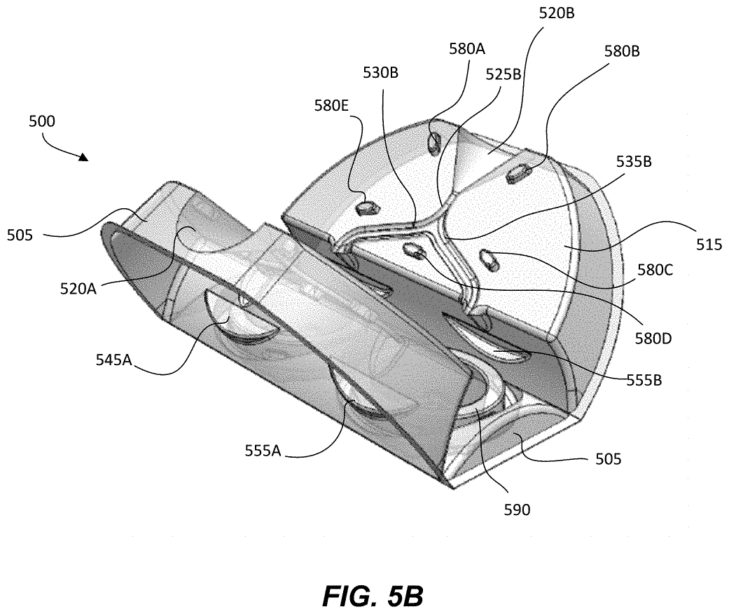

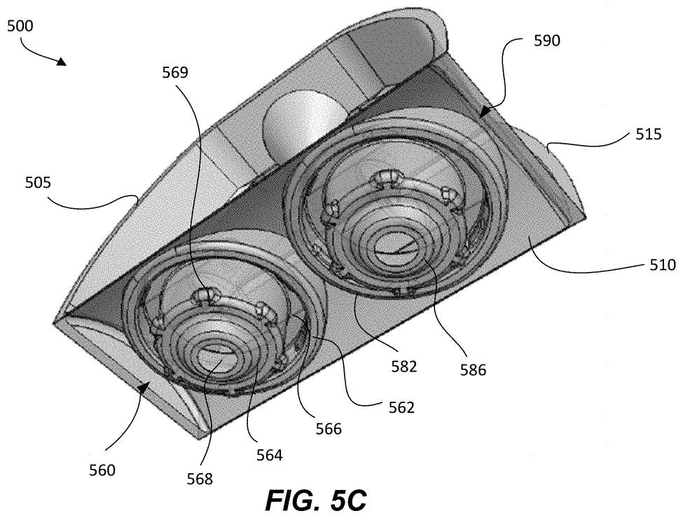

FIGS. 5A-5C illustrate multiple perspective views of an apparatus for separating a sample fluid. In some embodiments, the apparatus 500 may include a tri-fold design. Specifically, the apparatus 500 includes a first-fold section 505, a middle-fold section 510, and a second-fold section 515. The middle-fold section 510 is disposed between the first-fold section 505 and the second-fold section 515. The apparatus 500 can be a unitary structure including each of the first-fold section 505, the middle-fold section 510, and the second-fold section 515. In some embodiments, the first-fold section 505 and the second-fold section 515 can be removably attached to the middle-fold section 510. For example, the first-fold section 505 and the second-fold section 515 can be attached to the middle-fold section 510 using a fastener, for example, a hinge, a latch, a connecting member, among others. The apparatus 500 can be formed from any suitable material that provides adequate flexibility. In some embodiments, the apparatus 500 can be produced from a polymer. For example, the apparatus 500 can be an injection-molded article comprising a polymer.

FIG. 5A shows the first-fold section 505 adjacent a first side of the middle-fold section 510 and the second-fold section 515 adjacent the second side of the middle-fold section 510. The first-fold section 505 may include one or more grooves on a top face 511 of the first-fold section 505. For example, the first-fold section 505 incudes grooves 575A, 575B, 575C, 575D, and 575E on the top face 511 of the first-fold section 505. The grooves 575A, 575B, 575C, 575D, and 575E extend below the top face 511 of the first-fold section 505. The second-fold section 515 includes protrusions 580A, 580B, 580C, 580D, and 580E on the top face 516 of the second-fold section 515. The protrusions 580A, 580B, 580C, 580D, and 580E are raised in relation to the top face 516 of the second-fold section 515. For example, the top face 516 of the second-fold section 515 may be substantially planar and the protrusions 580A, 580B, 580C, 580D, and 580E may be raised in relation to the top face 516. The grooves 575A, 575B, 575C, 575D, and 575E on the top face 511 of the first-fold section 505 are configured to receive protrusions 580A, 580B, 580C, 580D, and 580E on the top face 516 of the second-fold section 515. The protrusions 580A, 580B, 580C, 580D, and 580E can be press fit into the respective grooves 575A, 575B, 575C, 575D, and 575E to secure the first-fold section 505 to the second-fold section 515. For example, protrusion 580A can mate with groove 575A, protrusion 580B can mate with groove 575B, protrusion 580C can mate with groove 575C, protrusion 580D can mate with groove 575D, and protrusion 580E can mate with groove 575E. In some embodiments, other types of fasteners can be used to attach the first-fold section 505 to the second-fold section 515. For example, the first-fold section 505 can be attached to the second-fold section 515 using gaskets, adhesives, latches, or other sealing mechanisms that provide a water-tight seal. The first-fold section 505 and the second-fold section 515 are configured to enclose the middle-fold section 510 when the first-fold section 505 and the second-fold section 515 are attached.

The top face 511 of the first-fold section 505 includes a portion of a receiving chamber 520A, a portion of a channel 525A, a portion of a first branch 530A, and a portion of a second branch 535A. The portion of the channel 525A extends from a distal end of the receiving chamber 520A. The portion of the channel 525A bifurcates into the portion of the first branch 530A and the portion of the second branch 535A. The top face 516 of the second-fold section 515 includes a second portion of the receiving chamber 520B, a second portion of the channel 525B, a second portion of the first branch 530B, and a second portion of the second branch 535B. The second portion of the channel 525B extends from a distal end of the receiving chamber 520B. The second portion of the channel 525B bifurcates into the second portion of the first branch 530B and the second portion of the second branch 535B.

The first portion of the receiving chamber 520A and the second portion of the receiving chamber 520B can extend into the plane of the top face to a depth. For example, first portion of the receiving chamber 520A can be a portion of a conical structure. The first portion of the receiving chamber 520A can be half of the conical structure taken along the Y-axis of the conical structure. In this regard, the width of the first portion of the receiving chamber 520A can increase from a first end to a second end. Similarly, the depth of the first portion of the receiving chamber 520A can increase from the first end to the second end. The distal end of the first portion of the receiving chamber 520A may be substantially U-shaped. The second portion of the receiving chamber 520B can have a corresponding geometry (e.g., the same shape and size) to form the receiving chamber.

In some embodiments, the top face 516 of the second-fold section 515 can be a mirror image of the top face 511 of the first-fold section 505. The top face 516 of the second-fold section 515 can be attached to the top face 511 of the first-fold section 505 to form a funnel. For example, the funnel includes the receiving chamber formed from the portion of a receiving chamber 520A and the second portion of the receiving chamber 520B, a channel extending from the distal end of the receiving chamber formed from the portion of the channel 525A and the second portion of the channel 525B, a first branch formed from the portion of the first branch 530A and the second portion of the first branch 530B, and a second branch formed from the portion of the second branch 535A and the second portion of the second branch 535B.

The first-fold section 505 includes a first interior-facing sidewall 540 and the second-fold section 515 includes a second interior-facing sidewall 550. The first interior-facing sidewall 540 includes a portion of a first through-hole 545A and a second through-hole 555A. The second interior-facing sidewall 550 includes a second portion of the first through-hole 545B and the second through-hole 555B. The portion of a first through-hole 545A on the first interior-facing sidewall 540 and the second portion of the first through-hole 545B on the second interior-facing sidewall 550 form the through hole to the first end cap 560 in the middle-fold section 510. The portion of a second through-hole 555A on the first interior-facing sidewall 540 and the second portion of the second through-hole 555B on the second interior-facing sidewall 550 form the through hole to the second end cap 590 in the middle-fold section 510. In some embodiments, each of the portions of the through holes can have a semi-circular shape. The portions of the through holes may each comprise an upper wall or lip 556 that is configured to be received by the middle-fold section 510. For example, the second portion of the second through-hole 545B on the second interior-facing sidewall 550 may include a lip 556 for sealing the region (e.g., annular groove 564) between the through hole and the first end cap 580. As shown in FIG. 5B, when the first-fold section 505 and the second-fold section 515 are attached, the first interior-facing sidewall 540 and the second interior-facing sidewall 550 form an arch or curved surface over the middle-fold section 510.

The first interior-facing sidewall 540 and the second interior-facing sidewall 550 extend from the top face of each of the first-fold section 505 and the second-fold section 515, respectively, to the middle-fold section 510. In some embodiments, the first interior-facing sidewall 540 and the second interior-facing sidewall 550 are concave. For example, the first interior-facing sidewall 540 and the second interior-facing sidewall 550 can be a curved wall that extends from an end of the top face of the first-fold section 505 and the second-fold section 515, respectively, to the middle-fold section 510. The first interior-facing sidewall 540 and the second interior-facing sidewall 550 includes a region that is connected to the middle-fold section 510. For example, the first interior-facing sidewall 540 and the second interior-facing sidewall 550 can be connected to the middle-fold section 510 via a hinge. The hinge is configured to allow the first interior-facing sidewall 540 of the first-fold section 505 and the second interior-facing sidewall 550 of the second-fold section 515 to move inward towards the middle-fold section 510. For example, FIG. 5B shows that each of the first interior-facing sidewall 540 of the first-fold section 505 and the second interior-facing sidewall 550 of the second-fold section 515 move inward towards the middle-fold section 510. The grooves 575A, 575B, 575C, 575D, and 575E on the first-fold section 505 receive the receive the protrusions 580A, 580B, 580C, 580D, and 580E on the second-fold section 515 to attach the first-fold section 505 to the second-fold section 515. The first-fold section 505 and the second-fold section 515 enclose the middle-fold section 510.

FIG. 5A shows the middle-fold section 510 disposed between the first-fold section 505 and the second-fold section 515. The middle-fold section 510 includes a contoured body including one or more end end caps. For example, as shown in FIG. 5A, the middle-fold section 510 may include a curved body. Each of the opposing ends of the middle-fold section 510 comprises an end wall 512, 514 that has a curvature that corresponds to the curvature of the first interior-facing sidewall 540 and the second interior-facing sidewall 550. In some embodiments, the curvature of the middle-fold section 510 corresponds to the curvature of the first interior-facing sidewall 540 and the second interior-facing sidewall 550 such that the apparatus forms a watertight seal as shown in FIG. 5B. For example, the concave surfaces of the first interior-facing sidewall 540 and the second interior-facing sidewall 550 can rest flush against the convex structure of the middle-fold section 510.

The middle-fold section 510 may include one or more end caps. For example, the middle-fold section 510 includes a first end cap 560 and a second end cap 580. The first end cap 560 includes an outer wall 562, an annular groove 564, and a cylindrical insert 566. The annular groove 564 is disposed between the outer wall 562 and the cylindrical insert 566. The annular groove 564 of each of the end caps can be sealed by the lip 556 on each of the through hole-portions (545A, 545B, 555A, and 555B). The outer wall 562 of the first end cap 560 has a height that varies around the circumference of the annular groove 564. For example, one or more regions of the outer wall 562 may have a peak height. In some embodiments, the two opposing ends of the outer wall can be bow-shaped, semicircular, or convex. In some embodiments, the outer wall 562 includes a set of opposing upper edges and can receive the opposing lip sections on the portions of the through-holes to enclose or seal the annular groove of the end caps. The second end cap 590 may have an identical structure as the first end cap 560. For example, the second end cap 590 includes an outer wall 592, a cylindrical insert 596, and an annular groove 594 disposed between the outer wall 592 and the cylindrical insert 596. The annular groove 594 can receive the upper wall or lip of 556 of the portions of the through-holes. For example, the lip of the first portion of the first through-hole 545A and the lip of the second portion of the first through-hole 545B can fit within the annular groove 564 of the first end cap 560.

FIG. 5C illustrates a bottom perspective view of the apparatus for splitting fluid. As discussed, the first-fold section 505 and the second-fold section 515 can be removably attached to form the apparatus 500 for receiving the sample fluid. Specifically, the first-fold section 505 and the second-fold section 515 include portions of the apparatus (e.g., half-portions) that combine to form the apparatus 500. The apparatus splits the fluid into two streams that can be deposited into two containers. The middle-fold section 510 includes the first end cap 560 and the second cap 590. Each of the first end cap 560 and the second cap 590 are configured to be attached a container, respectively. The first end cap 560 includes a cylindrical insert member 566 that is configured to extend into a depth of the container opening. The cylindrical insert member 566 may include a bottom wall that tapers to an orifice 568. For example, the bottom wall of the cylindrical insert 566 can be a truncated cone that includes an orifice 568 at a distal end for depositing a sample fluid directly within a container. In some embodiments, the cylindrical insert member 566 includes a side wall comprising a plurality of protrusions 569. The protrusions 569 can abut the interior wall of a container mouth for a secure fit to the container. The second end cap 590 also includes a cylindrical insert 596 that is configured to extend into a depth of the container opening with bottom wall that tapers to an orifice.

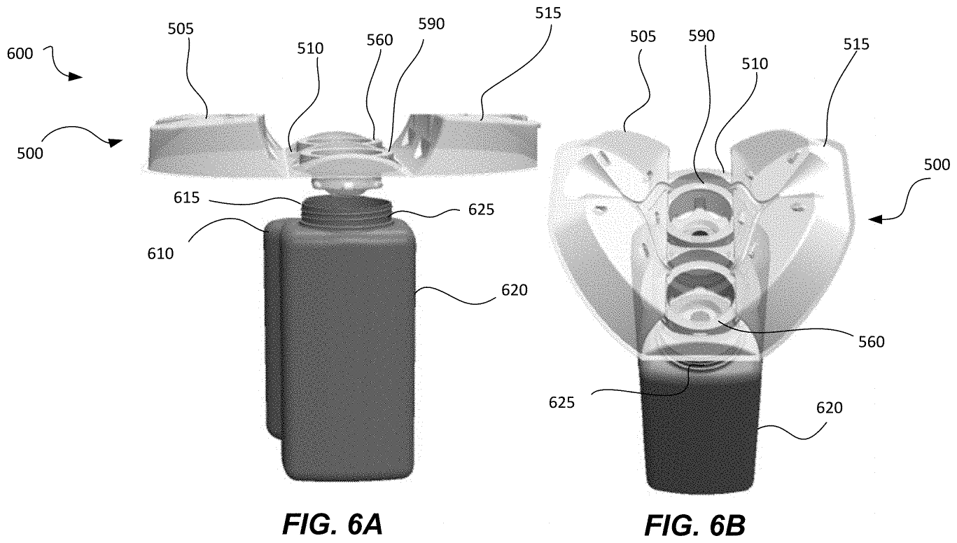

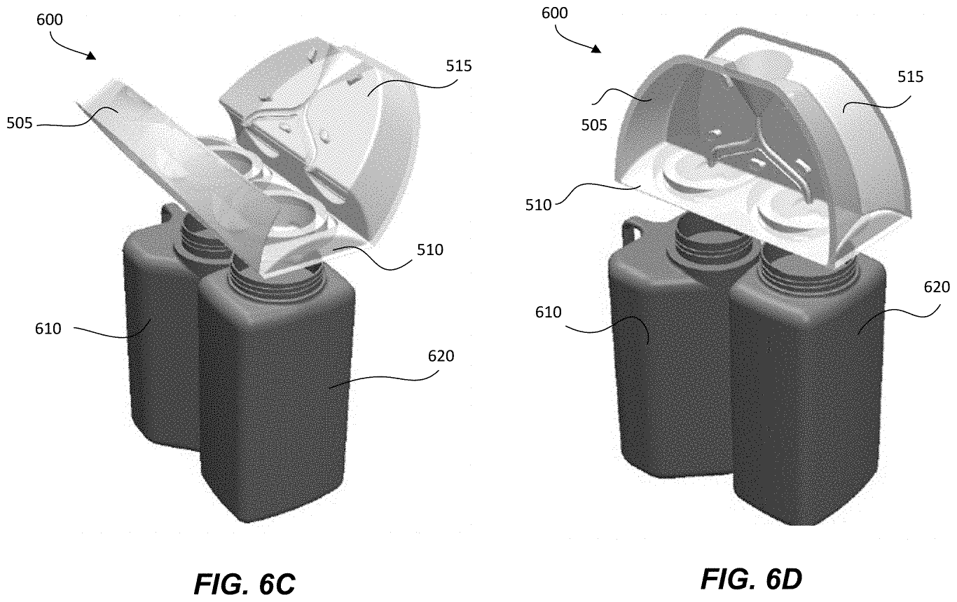

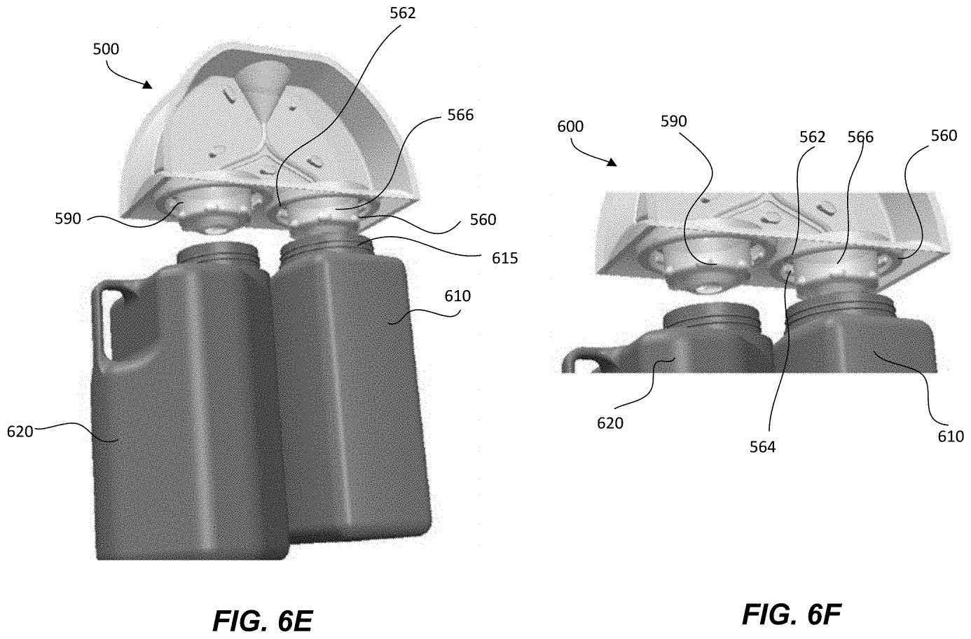

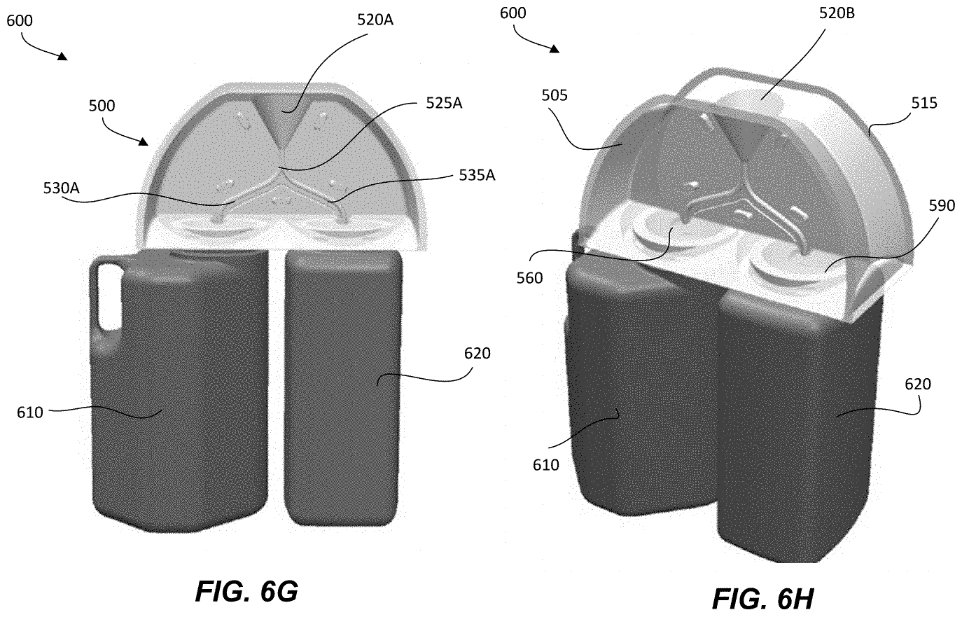

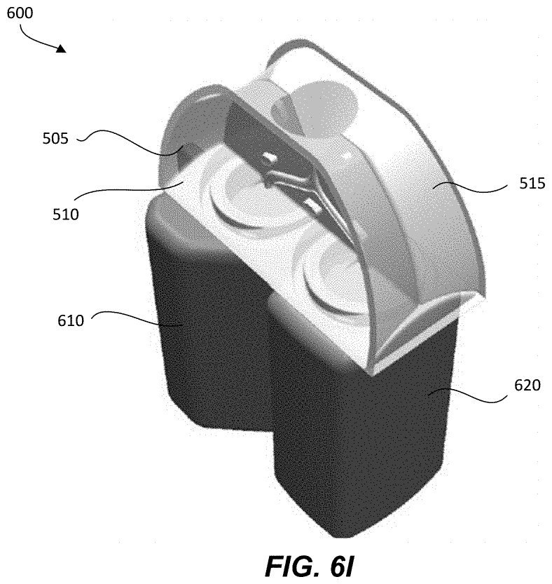

FIGS. 6A-6I show a system for collecting a sample fluid 600. For example, the system 600 may separate a sample (e.g., urine) into two containers for further downstream processing. The system 600 may include the apparatus 500 for separating a sample fluid discussed in FIGS. 5A-5C. For example, the system 600 may include the apparatus 500 including a tri-fold design. The system 600 includes a plurality of containers. In the embodiment shown in FIG. 6A, the system 600 includes a first container 610 and a second container 620. The first container 610 and the second container 620 can be removably attached to one another. For example, first container 610 and the second container 620 can be a unitary structure including a perforation to separate the containers when a force is applied. In some embodiments, first container 610 and a second container 620 can be fastened together using a fastener (e.g., a latch, Velcro, a lock, adhesive, etc.). In some embodiments, the first container 820 and the second container 830 are two separate containers that are not attached.

As shown in FIGS. 6A and 6B, the middle-fold section 510 of the apparatus 500 can be disposed over the first container 610 and the second container 620. For example, the first end cap 560 may be disposed over the mouth 615 of the first container 610 and the second end cap 590 may be disposed over the mouth 625 of the second container 620. The outer wall of the first end cap 560 and the second end cap 590 are disposed over the exterior surface of the mouth of the container. In this way, the cylindrical insert of the first end cap 560 and the second end cap 590 extends into the body of the container through the mouth. FIG. 6B shows that the outer wall of the first end cap 560 and the second end cap 590 extend over the sidewalls of the mouth and the orifice of the first end cap 560 and the second end cap 590 is within the body of the containers.

FIG. 6A shows the apparatus 500 in a first configuration. In some embodiments, the first end cap 560 and the second end cap 590 of the apparatus 500 can be disposed over the mouth of the container in a first configuration. In the first configuration, the first-fold section 505 and the second-fold section 515 of the apparatus 500 are substantially flat and horizontal with the middle-fold section 510. FIGS. 6B and 6C show each of the first-fold section 505 and the second-fold section 515 bent towards the middle-fold section 510. The first-fold section 505 and the second-fold section 515 are configured to move towards the middle-fold section 510 via a hinge connection. The first-fold section 505 and the second-fold section 515 are configured to enclose the middle-fold section 510. For example, FIG. 6D shows the first-fold section 505 and the second-fold section 515 enclose the region above the middle-fold section 510 in a second configuration. In the second configuration, the protrusions on the first-fold section 505 can mate with the grooves in the second-fold section 515 to attach the two sections. The first-fold section 505 and the second-fold section 515 completely enclose the area above the middle-fold section 510 to provide a watertight seal.

FIGS. 6E and 6F illustrate the cylindrical member 566 of the first end cap 560 being inserted into the mouth of the first container 610. The first end cap 560 and the second end cap 590 each include a cylindrical insert having an orifice at a distal end thereof that extends below the bottom surface of the middle-fold section 510 and into the body of the container. FIGS. 6E and 6F show that the cylindrical member 566 of first end cap 560 can be inserted into the body of the first container 610. The outer wall 562 of the first end cap 560 may abut or be adjacent to the exterior surface of the mouth 615 such that the wall of the mouth fits within the annular groove 564 of the first end cap 560. FIG. 6F shows a close-up view of the annular groove 564 of the first end cap 560 that receives the mouth 615 of the container.

FIG. 6G-I show the apparatus 500 attached to each of the first container 610 and the second container 620. As discussed herein, the top face of the second-fold section 515 can be attached to the top face of the first-fold section 505 to form a funnel. The funnel includes the receiving chamber formed from the portion of a receiving chamber 520A and the second portion of the receiving chamber 520B, a channel extending from the distal end of the receiving chamber formed from the portion of the channel 525A and the second portion of the channel 525B (not shown), a first branch formed from the portion of the first branch 530A and the second portion of the first branch 530B (not shown), and a second branch formed from the portion of the second branch 535A and the second portion of the second branch 535B (not shown). The first-fold section 505 and the second-fold section 515 enclose the middle-fold section 510 and provide through holes to the first end cap 560 and the second end cap 590. A sample fluid can be provided to the receiving chamber of the apparatus 500 to separate the fluid sample into the first container 610 and the second container 620. In some embodiments, the sample fluid is any biological fluid (e.g., blood, urine, separated blood, etc.). In this way, the fluid sample provided to the apparatus 500 can be separated into two portions that are provided through the first branch and the second branch to the first container 610 and the second container 620.

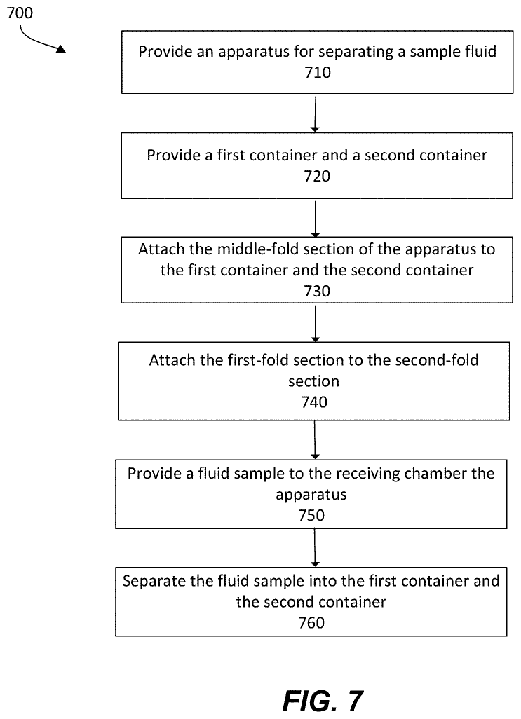

FIG. 7 provides a flow diagram of a method of separating a fluid sample according to some embodiments. The method 700 may include providing an apparatus for separating a sample fluid 710. The apparatus can be any apparatus described herein. For example, the apparatus described in FIGS. 5A-5C including a trifold design. The apparatus may include a first-fold section, a middle-fold section, and a second-fold section. The first-fold section and the second-fold section are configured to move inward relative to the middle-fold section to substantially enclose the middle-fold section. The first-fold section and the second-fold section each include a portion of a receiving chamber for receiving a sample fluid. The first-fold section and the second-fold section each include a portion of a channel that extends from the receiving chamber and bifurcates into a first branch and a second branch. The first branch and the second branch extend to a first through-hole and a second-through hole, respectively. In use, a sample fluid is supplied to the receiving chamber and is separated (e.g., equally) into the first branch and the second branch to the first through-hole and the second-through hole, respectively. The first through-hole and the second-through hole are disposed over a first end cap and a second end cap, respectively, of the middle-fold section. The first end cap and the second end cap each include a cylindrical insert having an orifice at distal end that extends below the bottom surface of the middle-fold section. The cylindrical insert is configured to be placed within the mouth of container to deposit the separated fluid sample in each respective container.

The method 700 may include providing a first container and a second container 720. In some embodiments, the first container and the second container can be any vessel that has an interior volume for retaining a fluid. For example, the first container and a second container can be urine specimen containers (e.g., 24-hour urine specimen containers). In some embodiments, the method may include removing a cap or enclosure of the container. For example, the urine specimen containers may include caps. The caps on the containers may be screw caps. The screw caps can be removably attached to the containers. In some embodiments, the containers may include one or more fluids that are added prior to receiving the sample fluid. For example, one of the containers may include a preservative. The preservative can be an acid-based preservative. For example, the first container may include acetic acid or hydrochloric acid.

The method 700 may include attaching the middle-fold section to the first container and the second container 730. In some embodiments, the first end cap of the middle-fold section can be disposed over a receiving region (e.g., the mouth of a container) of the first container. Similarly, the second end cap of the middle-fold section can be disposed over a receiving region of the second container. The first end cap and the second end cap each include a cylindrical insert having an orifice at a distal end thereof that extends below the bottom surface of the middle-fold section and into the body of the container.

The method 700 may include attaching the first-fold section to the second-fold section 740. For example, the first-fold section and the second-fold section can be bent inward toward the middle-fold section. The first-fold section and the second-fold section are configured to move inward relative to the middle-fold section to substantially enclose the middle-fold section. The first-fold section and the second-fold section each form a receiving chamber for receiving a sample fluid and a channel at a distal end of the receiving chamber. The channel separates into a first branch and a second branch that provides a sample fluid through a first through-hole and a second-though hole, respectively, to the first end cap and the second end cap.

The method 700 may include providing a sample fluid to the receiving chamber of the apparatus 750. In some embodiments, the sample fluid is any biological fluid (e.g., blood, urine, separated blood, etc.). The method 700 includes separating the fluid sample into the first container and the second container 760. In this way, the fluid sample provided to the apparatus can be separated into two portions that are provided through the first branch and the second branch to the first container and the second container. In some embodiments, the method may include collecting a fluid sample and then providing the fluid sample to the receiving chamber of the apparatus. The fluid sample may be a urine sample. The urine sample can be collected from a patient and poured into the receiving chamber of the apparatus. The urine sample travels from the receiving chamber to the channel. The urine sample is split in equal proportions into the first branch and the second branch to fill the first container and the second container via the end caps. In some embodiments, the method includes removing the apparatus the fluid sample is separated and collected. In some embodiments, the caps can be replaced with a tamper-resistant seal cap.

### Illustrations

Illustration 1: An apparatus for separating a fluid sample, the apparatus comprising: a receiving chamber comprising a first opening and a second opening, wherein the first opening has a larger diameter than the second opening; a channel extending from the second opening of the receiving chamber, wherein the channel comprises a proximal end adjacent the receiving chamber and a distal end; wherein the distal end of the channel bifurcates into a first branch and a second branch.

Illustration 2: The illustration of any preceding or subsequent illustration, wherein the receiving chamber comprises a truncated cone, wherein the truncated cone has an interior volume configured to receive the fluid sample.

Illustration 3: The illustration of any preceding or subsequent illustration, wherein the diameter of the receiving chamber continually decreases from the first opening to the second opening.

Illustration 4: The illustration of any preceding or subsequent illustration, wherein the first branch and the second branch extend in opposing directions from the channel.

Illustration 5: The illustration of any preceding or subsequent illustration, wherein the first branch and the second branch are disposed at an acute angle from a long axis of the channel.

Illustration 6: The illustration of any preceding or subsequent illustration, wherein a diameter of the channel from the proximal end to the distal end is constant.

Illustration 7: The illustration of any preceding or subsequent illustration, wherein the distal end of the channel, the first branch, and the second branch form a junction, wherein the junction is configured to separate the fluid sample into the first branch and the second branch.

Illustration 8: The illustration of any preceding or subsequent illustration, wherein a diameter of the first branch and the second branch is less than the diameter of the channel.

Illustration 9: The illustration of any preceding or subsequent illustration, wherein the fluid sample comprises urine.

Illustration 10: The illustration of any preceding or subsequent illustration, wherein the apparatus includes a first leg extending from the first branch and a second leg extending from the second branch.

Illustration 11: The illustration of any preceding or subsequent illustration, wherein the first leg and the second leg are parallel to the channel.

Illustration 12: The illustration of any preceding or subsequent illustration, wherein the apparatus includes a first cap for receiving the first branch and a second cap for receiving the second branch.

Illustration 13: The illustration of any preceding or subsequent illustration, wherein the first cap and the second cap each comprise an orifice configured to receive the first branch and the second branch.

Illustration 14: The illustration of any preceding or subsequent illustration, wherein the first cap and the second cap include a plastic disposed over the orifice, wherein the plastic provides a seal when the first branch and the second branch are each inserted into the first cap and the second cap through the orifice.

Illustration 15: The illustration of any preceding or subsequent illustration, wherein the first cap and the second cap are self-sealing caps optionally fitted to attach to and secure the apparatus to a first orifice on a first container and a second orifice on a second container, respectively.

Illustration 16: A system for separating a fluid sample, the system comprising: an apparatus comprising: a receiving chamber comprising a first opening and a second opening, wherein the first opening has a larger diameter than the second opening; a channel extending from the second opening of the receiving chamber, wherein the channel comprises a proximal end adjacent the receiving chamber and a distal end; and wherein the distal end of the channel bifurcates into a first branch and a second branch; a first container comprising a first cap; and a second container comprising a second cap.

Illustration 17: The illustration of any preceding or subsequent illustration, wherein the diameter of the receiving chamber continually decreases from the first opening to the second opening.

Illustration 18: The illustration of any preceding or subsequent illustration, wherein the first branch and the second branch are disposed at an acute angle from a long axis of the channel.

Illustration 19: The illustration of any preceding or subsequent illustration, wherein a diameter of the channel from the proximal end to the distal end is constant.

Illustration 20: The illustration of any preceding or subsequent illustration, wherein the distal end of the channel, the first branch, and the second branch form a junction, wherein the junction is configured to separate the fluid sample into the first branch and the second branch.

Illustration 21: The illustration of any preceding or subsequent illustration, wherein the apparatus further comprises a first leg extending from the first branch and a second leg extending from the second branch.

Illustration 22: The illustration of any preceding or subsequent illustration, wherein the first leg and the second leg are parallel to the channel.

Illustration 23: The illustration of any preceding or subsequent illustration, wherein the first cap and the second cap each comprise an orifice configured to receive the first leg and the second leg.

Illustration 24: The illustration of any preceding or subsequent illustration, wherein the first cap and the second cap include a plastic disposed over the orifice, wherein the plastic provides a seal when the first leg and the second leg are each inserted into the first cap and the second cap through the orifice.

Illustration 25: The illustration of any preceding or subsequent illustration, wherein the first cap and the second cap are self-sealing caps optionally fitted to attach to and secure the apparatus to a first orifice on the first container and a second orifice on the second container, respectively.

Illustration 26: A method for separating a fluid sample, the method comprising: providing an apparatus comprising: a receiving chamber comprising a first opening and a second opening, wherein the first opening has a larger diameter than the second opening; a channel extending from the second opening of the receiving chamber, wherein the channel comprises a proximal end adjacent the receiving chamber and a distal end; and wherein the distal end of the channel bifurcates into a first branch and a second branch; providing a first container and a second container, wherein the first container comprises a first cap and the second container comprises a second cap; inserting the first branch through the first cap into the first container and the second branch through the second cap into the second container; and providing a fluid sample to the receiving chamber of the apparatus.

Illustration 27: The illustration of any preceding or subsequent illustration, wherein inserting the first branch into the first cap and inserting the second branch into the second cap comprises puncturing a sealing film disposed over an orifice of the first cap and the second cap.

Illustration 28: The illustration of any preceding or subsequent illustration, wherein the method includes collecting a fluid sample.

Illustration 29: The illustration of any preceding or subsequent illustration, wherein the fluid sample is separated into the first container via the first branch and the second container via the second branch.

Illustration 30: The illustration of any preceding or subsequent illustration, wherein the method includes removing the first branch and the second branch from the first cap and the second cap after the fluid sample is collected.

Illustration 31: The illustration of any preceding or subsequent illustration, wherein the method includes replacing each of the first cap and the second cap with a tamper-resistant seal cap.

Illustration 32: An apparatus for separating a fluid sample, the apparatus comprising: a first-fold section comprising: a portion of a receiving chamber; a portion of a channel extending from a distal end of the receiving chamber, wherein the channel bifurcates into a portion of a first branch and a second branch; a second-fold section comprising: a second portion of the receiving chamber; a second portion of the channel extending from a distal end of the receiving chamber, wherein the channel bifurcates into a second portion of the first branch and the second branch; and a middle-fold section disposed between the first-fold section and the second-fold section, wherein the middle-fold section comprises a first end cap and a second end cap; wherein the first-fold section and the second-fold section are configured to fold inward toward the middle-fold section.

Illustration 33: The illustration of any preceding or subsequent illustration, further comprising: a first hinge between the first-fold section and the middle-fold section; and a second hinge between the second-fold section and the middle-fold section.

Illustration 34: The illustration of any preceding or subsequent illustration, wherein the first-fold section is configured to be removably attached to the second-fold section.

Illustration 35: The illustration of any preceding or subsequent illustration, wherein the portion of the receiving chamber of the first-fold section corresponds to the second portion of the receiving chamber of the second-fold section to form the receiving chamber, the portion of the channel of the first-fold section corresponds to the second portion of the channel of the second-fold section to form the channel, and the portion of the first branch and second branch of the first-fold section corresponds to the second portion of the first branch and second branch of the second-fold section to form the first branch and second branch.

Illustration 36: The illustration of any preceding or subsequent illustration, wherein the portion of the receiving chamber of the first-fold section and the second portion of the receiving chamber of the second-fold section increase in depth and width from a first end to a second end.

Illustration 37: The illustration of any preceding or subsequent illustration, wherein the first-fold section and the second-fold section comprise an interior-facing sidewall extending from a top face to the middle-fold section.

Illustration 38: The illustration of any preceding or subsequent illustration, wherein the interior-facing sidewall is curved.

Illustration 39: The illustration of any preceding or subsequent illustration, wherein the interior-facing sidewall of the first-fold section includes a portion of a first through hole and a second through hole, wherein the interior-facing sidewall of the second-fold section includes a second portion of the first through hole and the second through hole.

Illustration 40: The illustration of any preceding or subsequent illustration, wherein each of the first end cap and the second end cap comprise: a cylindrical insert member comprising an orifice; an annular groove surrounding the cylindrical insert member; and an outer wall.

Illustration 41: The illustration of any preceding or subsequent illustration, wherein the cylindrical insert member includes bottom region that tapers to the orifice.

Illustration 42: The illustration of any preceding or subsequent illustration, wherein the middle-fold section comprises a hemispherical body.

Illustration 43: The illustration of any preceding or subsequent illustration, wherein a top face of the first-fold section includes a plurality of grooves and a top face of the second-fold section includes a plurality of protrusions.

Illustration 44: A system for separating a fluid sample, the system comprising: an apparatus comprising: a first-fold section comprising: a portion of a receiving chamber; a portion of a channel extending from a distal end of the receiving chamber, wherein the channel bifurcates into a portion of a first branch and a second branch; a second-fold section comprising: a second portion of the receiving chamber; a second portion of the channel extending from a distal end of the receiving chamber, wherein the channel bifurcates into a second portion of the first branch and the second branch; and a middle-fold section disposed between the first-fold section and the second-fold section, wherein the middle-fold section comprises a first end cap and a second end cap; wherein the first-fold section and the second-fold section are configured to fold inward toward the middle-fold section to enclose the middle-fold section; a first container;

and a second container.

Illustration 45: The illustration of any preceding or subsequent illustration, wherein each of the first end cap and the second end cap comprise: a cylindrical insert member comprising an orifice; an annular groove surrounding the cylindrical insert member; and an outer wall.

Illustration 46: The illustration of any preceding or subsequent illustration, wherein the cylindrical insert member includes a bottom wall that tapers to the orifice. In some embodiments, the cylindrical insert member is configured to be inserted within the first container and the second container.

Illustration 47: The illustration of any preceding or subsequent illustration, wherein the apparatus further comprises: a first hinge between the first-fold section and the middle-fold section; and a second hinge between the second-fold section and the middle-fold section.

Illustration 48: The illustration of any preceding or subsequent illustration, wherein a top face of the first-fold section includes a plurality of grooves and a top face of the second-fold section includes a plurality of protrusions.

Illustration 49: The illustration of any preceding or subsequent illustration, wherein the first-fold section and the second-fold section comprise an interior-facing sidewall extending from a top face to the middle-fold section, wherein the interior-facing sidewall is curved.

Illustration 50: A method for separating a fluid sample, the method comprising: providing an apparatus comprising: a first-fold section comprising: a portion of a receiving chamber; a portion of a channel extending from a distal end of the receiving chamber, wherein the channel bifurcates into a portion of a first branch and a second branch; a second-fold section comprising: a second portion of the receiving chamber; a second portion of the channel extending from a distal end of the receiving chamber, wherein the channel bifurcates into a second portion of the first branch and the second branch; and a middle-fold section disposed between the first-fold section and the second-fold section, wherein the middle-fold section comprises a first end cap and a second end cap; wherein the first-fold section and the second-fold section are configured to fold inward toward the middle-fold section to enclose the middle-fold section; providing a first container and a second container; attaching the first end cap to the first container; attaching the second end cap to the second container; attaching the first-fold section to the second-fold section; providing a fluid sample to the receiving chamber of the apparatus; and separating the fluid into the first container and the second container.

Illustration 51: The illustration of any preceding or subsequent illustration, wherein attaching the first end cap to the first container comprises inserting a cylindrical insert member of the first end cap into a mouth of the first container.

Illustration 52: The illustration of any preceding or subsequent illustration, wherein the method further comprises collecting a fluid sample.

Illustration 53: The illustration of any preceding or subsequent illustration, wherein the sample is separated into the first container via the first branch and the second container via the second branch.

Illustration 54: The illustration of any preceding or subsequent illustration, wherein the method further comprises removing the apparatus after the fluid sample is collected.

Illustration 55: The illustration of any preceding or subsequent illustration, wherein the method further comprises sealing the first container and the second container.

All patents, publications and abstracts cited above are incorporated herein by reference in their entireties. Various embodiments of the invention have been described in fulfillment of the various objectives of the invention. It should be recognized that these embodiments are merely illustrative of the principles of the present invention. Numerous modifications and adaptions thereof will be readily apparent to those skilled in the art without departing from the spirit and scope of the present invention as defined in the following claims.

What is claimed is:

1. An apparatus for separating a fluid sample, the apparatus comprising:
a receiving chamber comprising a first opening and a second opening, wherein the first opening has a larger diameter than the second opening;
a channel extending from the second opening of the receiving chamber, wherein the channel comprises a proximal end adjacent the receiving chamber and a distal end;
wherein the distal end of the channel bifurcates into a first branch and a second branch.

2. The apparatus of claim 1, wherein the receiving chamber comprises a truncated cone, wherein the truncated cone has an interior volume configured to receive the fluid sample.

3. The apparatus of claim 1, wherein the diameter of the receiving chamber continually decreases from the first opening to the second opening.

4. The apparatus of claim 1, wherein the first branch and the second branch extend in opposing directions from the channel.

5. The apparatus of claim 1, wherein the first branch and the second branch are disposed at an acute angle from a long axis of the channel.

6. The apparatus of claim 1, wherein a diameter of the channel from the proximal end to the distal end is constant.

7. The apparatus of claim 1, wherein the distal end of the channel, the first branch, and the second branch form a junction, wherein the junction is configured to separate the fluid sample into the first branch and the second branch.

8. The apparatus of claim 1, wherein a diameter of the first branch and the second branch is less than the diameter of the channel.

9. The apparatus of claim 1, wherein the fluid sample comprises urine.

10. The apparatus of claim 1, further comprising a first leg extending from the first branch and a second leg extending from the second branch.

11. The apparatus of claim 10, wherein the first leg and the second leg are parallel to the channel.

12. The apparatus of claim 1, further comprising a first cap for receiving the first branch and a second cap for receiving the second branch.

13. The apparatus of claim 12, wherein the first cap and the second cap each comprise an orifice configured to receive the first branch and the second branch.

14. The apparatus of claim 13, wherein the first cap and the second cap include a plastic disposed over the orifice, wherein the plastic provides a seal when the first branch and the second branch are each inserted into the first cap and the second cap through the orifice.

15. The apparatus of claim 13, wherein the first cap and the second cap are self-sealing caps optionally fitted to attach to and secure the apparatus to a first orifice on a first container and a second orifice on a second container, respectively.

16. A system for separating a fluid sample, the system comprising:
an apparatus comprising:
a receiving chamber comprising a first opening and a second opening, wherein the first opening has a larger diameter than the second opening;
a channel extending from the second opening of the receiving chamber, wherein the channel comprises a proximal end adjacent the receiving chamber and a distal end; and
wherein the distal end of the channel bifurcates into a first branch and a second branch;
a first container comprising a first cap; and
a second container comprising a second cap.

17. The system of claim 16, wherein the diameter of the receiving chamber continually decreases from the first opening to the second opening.

18. The system of claim 16, wherein the first branch and the second branch are disposed at an acute angle from a long axis of the channel.

19. The system of claim 16, wherein a diameter of the channel from the proximal end to the distal end is constant.

20. The system of claim 16, wherein the distal end of the channel, the first branch, and the second branch form a junction, wherein the junction is configured to separate the fluid sample into the first branch and the second branch.

21. The system of claim 16, wherein the apparatus further comprises a first leg extending from the first branch and a second leg extending from the second branch.

22. The system of claim 21, wherein the first leg and the second leg are parallel to the channel.

23. The system of claim 22, wherein the first cap and the second cap each comprise an orifice configured to receive the first leg and the second leg.

24. The system of claim 23, wherein the first cap and the second cap include a plastic disposed over the orifice, wherein the plastic provides a seal when the first leg and the second leg are each inserted into the first cap and the second cap through the orifice.

25. The system of claim 23, wherein the first cap and the second cap are self-sealing caps optionally fitted to attach to and secure the apparatus to a first orifice on the first container and a second orifice on the second container, respectively.

26. A method for separating a fluid sample, the method comprising:
providing an apparatus comprising:
a receiving chamber comprising a first opening and a second opening, wherein the first opening has a larger diameter than the second opening;
a channel extending from the second opening of the receiving chamber, wherein the channel comprises a proximal end adjacent the receiving chamber and a distal end; and
wherein the distal end of the channel bifurcates into a first branch and a second branch;
providing a first container and a second container, wherein the first container comprises a first cap and the second container comprises a second cap;
inserting the first branch through the first cap into the first container and the second branch through the second cap into the second container; and
providing a fluid sample to the receiving chamber of the apparatus.

27. The method of claim 26, wherein inserting the first branch into the first cap and inserting the second branch into the second cap comprises puncturing a sealing film disposed over an orifice of the first cap and the second cap.

28. The method of claim 26, further comprising collecting a fluid sample.

29. The method of claim 26, wherein the fluid sample is separated into the first container via the first branch and the second container via the second branch.

30. The method of claim 26, further comprising removing the first branch and the second branch from the first cap and the second cap after the fluid sample is collected.

31. The method of claim 30, further comprising replacing each of the first cap and the second cap with a tamper-resistant seal cap.
